(19) United States
(12) Patent Application Publication
(10) Pub. No.: US 2018/0052081 A1
(43) Pub. Date: Feb. 22, 2018

(54) COMBINING MODIFIED ANTIBODIES WITH EXPANSION MICROSCOPY FOR IN-SITU, SPATIALLY-RESOLVED PROTEOMICS

(71) Applicant: Expansion Technologies, Boston, MA (US)

(72) Inventor: Richie E. KOHMAN, Cambridge, MA (US)

(73) Assignee: Expansion Technologies, Boston, MA (US)

(21) Appl. No.: 15/592,221

(22) Filed: May 11, 2017

Related U.S. Application Data
(63) Continuation of application No. 62/334,628, filed on May 11, 2016.

(51) Int. Cl. G01N 1/30; G01N 33/533; G01N 33/543; G01N 33/545

(52) U.S. Cl. CPC G01N 1/30; G01N 33/545; G01N 33/54306; G01N 33/533

(57) ABSTRACT

This invention relates to imaging, such as by expansion microscopy, labelling, and analyzing biological samples, such as cells and tissues, as well as reagents and kits for doing so.

### CROSS REFERENCE TO RELATED APPLICATIONS

This application claims the benefit of U.S. Provisional Application No. 62/334,628, filed on May 11, 2016, which is incorporated by refereince herein in its entirety.

### FIELD OF THE INVENTION

The invention relates to imaging, such as by expansion microscopy, labelling, and analyzing biological samples, such as cells and tissues, as well as reagents and kits for doing so.

### BACKGROUND OF THE INVENTION

In expansion microscopy (ExM), 3-dimensional imaging with nanoscale precision is performed on cells and tissues. This is accomplished by physically expanding the biological sample using a dense polymer matrix (FIG. 1). The first step of this process involves treating the tissue with a fluorescent protein-binding-group (typically an antibody) that selectively binds to the protein being analyzed. Next the sample is infused with a monomer solution that permeates into the tissue. Free radical polymerization of this solution creates a polymer network that is physically connected to the protein-binding-groups through customized bioconjugation chemistry. Lastly, the tissue is digested and the hydrogel (and fluorescent dyes) expands uniformly. The result is a polymer network that contains fluorescent dyes where the target proteins were located. This process has many advantages. Notably, it allows pseudo super-resolution imaging with conventional confocal microscopy because the imaging targets are no longer diffraction limited. Additionally, the tissue digestion clears the sample allowing imaging deep into thick tissues samples. (See, e.g., Le Goff et al., Eur. Polym. J. [2015], http://dx.doi.org/10.1016/j.eurpolymj.2015.02.022)

Critical to the success of the ExM process is the ability to physically connect the fluorescent protein-binding-groups to the polymer network. Current ExM attachment chemistry uses a trifunctional, double-stranded DNA linker to accomplish this. Because the tissue digestion enzymes are also capable of digesting the antibodies typically used as protein-binding-groups, the fluorescent dyes must be attached to the DNA and not the antibody. Also needed is the presence of a chemical group that can polymerize into the gel matrix. Current examples of ExM use the chemical arrangement shown in FIG. 2 where one strand of DNA is connected to the protein-binding-group while the complementary strand possesses both the dye and the polymerizable group. Using this strategy , cells and brain tissue were successfully stained with up to 3 different protein-binding-groups, expanded, and imaged (Chen et al., Science 347:543(2015)). However, because the number of fluorescent dyes that can be used is small (typically <6), this strategy is limited to imaging only a small number of proteins per sample. Additionally, the polymerization process dampens the fluorescence of the dyes, which are permanently connected to the gel matrix. An alternative bioconjugation strategy has also been utilized to overcome some of these drawbacks. By rearranging the location of the three chemical groups (dye, gel binding group, and protein-binding-group) on the DNA linker, some previous limitations in protein imaging have been overcome.

However, using DNA/antibody conjugates has several disadvantages. Buffers with uncommon additives are necessary in order to prevent the DNA on the antibody from binding to the nuclear DNA in the sample. Also, the presence of the DNA on the antibody reduces the extent and the rate at which it binds to the target. The result is that the current ExM processes are lengthy, and the staining is commonly dim compared to controls. Surprisingly, it has been found not only that antibodies can be directly acrylated (and hence suitable for polymerization), either before, after, or at the same time as attachment with a detectable label, but also that the detectable label will remain after the tissue digestion step, which is necessary for ExM. Because the ExM digestion enzyme can possibly degrade antibodies, it had been assumed previously that all label detection would disappear with digestion. However, the present invention, turbo-expansion microscopy (TurboExM), does not use DNA as a linker, stains samples brightly, and is more rapid than previous ExM processes.

### SUMMARY OF THE INVENTION

In one aspect, provided herein are methods of labeling a biological sample, the methods comprising: contacting the sample with at least one binding composition under conditions where it selectively recognizes a target biomolecule, wherein the binding composition comprises a modified antibody comprising a first antibody having an antigen-binding site having an affinity for the target biomolecule, wherein the first antibody is operably linked to (i) a detectable label, and (ii) a polyelectrolyte gel binding moiety; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; and dialyzing the sample to expand the polyelectrolyte gel. In some embodiments, the methods further comprise the step of removing the binding composition unbound to the polyelectrolyte gel after covalently conjugating the polyelectrolyte gel binding moiety to it. In some embodiments, the label and/or the polyelectrolyte gel binding moiety is operably linked to a constant region of the first antibody. In some embodiments, the label and/or the polyelectrolyte gel binding moiety is operably linked at the location of one or more disulfide linkages with the antibody. In some embodiments, the label and the polyelectrolyte gel binding moiety are operably linked to the first antibody either simultaneously or sequentially in either order.

In another aspect, provided herein are methods of labeling a biological sample, the methods comprising: contacting the sample with at least one binding composition under conditions where it selectively recognizes a target biomolecule, wherein the binding composition comprises a modified antigen-binding fragment comprising a first antigen-binding fragment having an antigen-binding site having an affinity for the target biomolecule, wherein the first antigen-binding fragment is operably linked to (i) a detectable label, and (ii) a polyelectrolyte gel binding moiety; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; and dialyzing the sample to expand the polyelectrolyte gel. In some embodiments, the methods further comprise the step of removing the binding composition unbound to the polyelectrolyte gel after covalently conjugating the polyelectrolyte gel binding moiety to it. In some embodiments, the label and/or the polyelectrolyte gel binding moiety is/are operably linked to a constant region of the first antigen-binding fragment. In some embodiments, the label and/or the polyelectrolyte gel binding moiety is operably linked at the location of one or more disulfide linkages with the antibody. In some embodiments, the label and the polyelectrolyte gel binding moiety are operably linked to the first antigen-binding fragment either simultaneously or sequentially in either order.

In another aspect, provided herein are methods of imaging a biological sample, the methods comprising labeling the sample via any one of the methods as described herein and obtaining an image of the sample after expanding the polyelectrolyte gel. In some embodiments, the methods of imaging a biological sample further comprise obtaining an image of the sample before expanding the polyelectrolyte gel.

In another aspect, provided herein are methods of analyzing a biological sample, the methods comprising the steps of: contacting the sample with a set of modified antibodies that selectively recognize a set of target biomolecules under conditions where the modified antibodies selectively recognize the target biomolecules, wherein each modified antibody comprises an antibody comprising an antigen-binding site having an affinity specific for one of the target biomolecules, wherein the antibody is operably linked to (i) a detectable label distinct to that antibody, and (ii) a polyelectrolyte gel binding moiety; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; dialyzing the sample to expand the polyelectrolyte gel; and for each target biomolecule, detecting the label distinct for the antibody having an affinity specific for that target biomolecule. In some embodiments, the label operably linked to the antibody for a plurality of the target biomolecules includes a fluorophore that is common for that plurality of targets, and each antibody of that plurality is hybridized separately from the other probes of that plurality and is removed following detection of that label. In some embodiments, the plurality of target biomolecules is the set of target biomolecules.

In another aspect, provided herein are methods of analyzing a biological sample, the methods comprising the steps of: contacting the sample with a set of modified antigen-binding fragments that selectively recognize a set of target biomolecules under conditions where the modified antigen-binding fragments selectively recognize the target biomolecules, wherein each modified antigen-binding fragment comprises an antigen-binding fragment comprising an antigen-binding site having an affinity specific for one of the target biomolecules, wherein the antigen-binding fragment is operably linked to (i) a detectable label distinct to that antigen-binding fragment, and (ii) a polyelectrolyte gel binding moiety; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; dialyzing the sample to expand the polyelectrolyte gel; and for each target biomolecule, detecting the label distinct for the antigen-binding fragment having an affinity specific for that target biomolecule. In some embodiments, the label operably linked to the antigen-binding fragment for a plurality of the target biomolecules includes a fluorophore that is common for that plurality of targets, and each antigen-binding fragment of that plurality is hybridized separately from the other probes of that plurality and is removed following detection of that label. In some embodiments, the plurality of target biomolecules is the set of target biomolecules.

In another aspect, provided herein are kits for modifying an antibody or an antigen-binding fragment, the kits comprising: a first reagent comprising a label; and a second reagent comprising a polyelectrolyte gel binding moiety. In some embodiments, the kits further comprise a first antibody or a first antigen-binding fragment, the first antibody or the first-antigen-binding fragment comprising an antigen-binding site having an affinity for a target biomolecule.

In another aspect, provided herein are kits for labeling a biological sample, the kit comprising: a first reagent comprising a label; a second reagent comprising a polyelectrolyte gel binding moiety; and [a protease. In some embodiments, the kits further comprise a first antibody or a first antigen-binding fragment, the first antibody or the first-antigen-binding fragment comprising an antigen-binding site having an affinity for a target biomolecule. In some embodiments, the kits further comprise monomers of a polyelectrolyte gel, a cross-linking reagent, a detection reagent specific for the label, and/or a physiological buffer.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIG. 1. Schematic depiction of tissue processing performed in expansion microscopy (ExM).

FIG. 2. General attachment strategy used for expansion microscopy.

FIG. 3. Schematic depiction of an example of antibody modification for turbo-expansion microscopy (TurboExM). The detectable label (here a fluorophore, shown as a sphere) can be attached before, after, or at the same time as the acrylamide group, which is attached here via direct acrylation.

FIG. 4. Comparison of mouse brain slices: The slice on the left was stained with primary anti-parvalbumin (Millipore MAB1572) antibody and a custom-made acrylated secondary antibody modified with SE, 6-((acryloyl)amino)hexanoic acid, succinimidyl ester (Acryoyl-X, ThermoFisher A20770) and labelled with the Atto 647N dye (Sigma 18373). The sample was polymerized, digested and expanded, as shown in the scale bar (left scale bar=1000 μm; right scale bar =5000 μm) to produce a sample which retained the staining pattern of the pre-expanded sample (right).

### DETAILED DESCRIPTION OF THE INVENTION

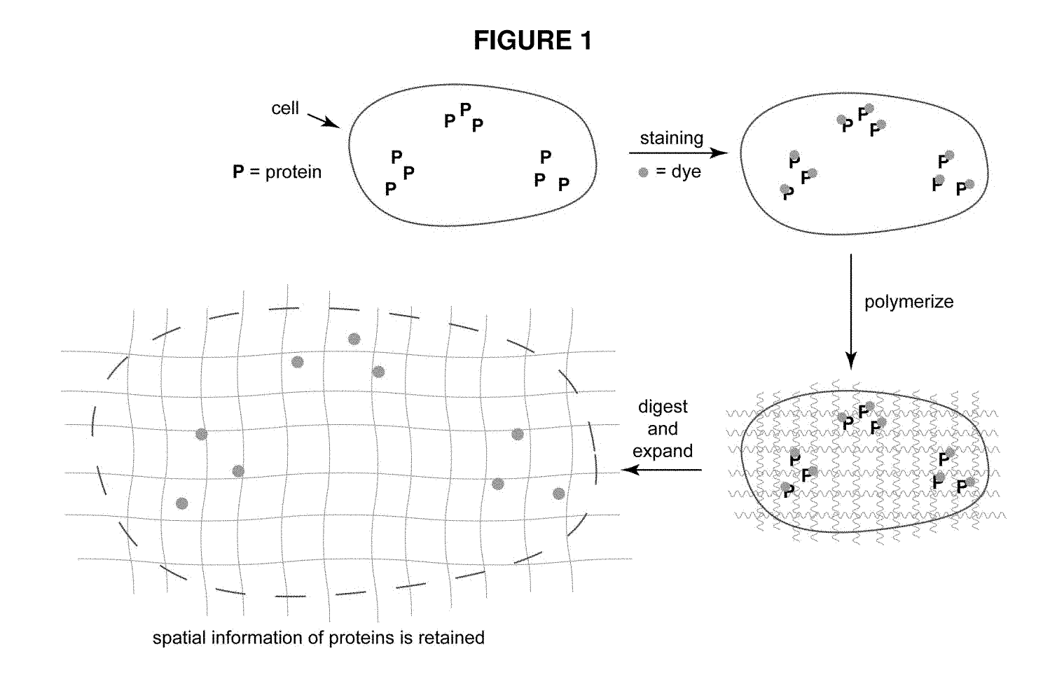

In expansion microscopy (ExM), 3-dimensional imaging with nanoscale precision is performed on cells and tissues. This is accomplished by physically expanding the biological sample using a dense polymer matrix (FIG. 1). The first step of this process involves treating the tissue with a fluorescent protein-binding-group (typically an antibody) that selectively binds to the protein being analyzed. Next the sample is infused with a monomer solution that permeates into the tissue. Free radical polymerization of this solution creates a polymer network that is physically connected to the protein-binding-groups through customized bioconjugation chemistry. Lastly, the tissue is digested and the hydrogel (and fluorescent dyes) expands uniformly. The result is a polymer network that contains fluorescent dyes where the target proteins were located. This process had many advantages. Notably, it allows pseudo super resolution imaging with conventional confocal microscopy because the imaging targets are no longer diffraction limited. Additionally, the tissue digestion clears the sample allowing imaging deep into thick tissues samples.

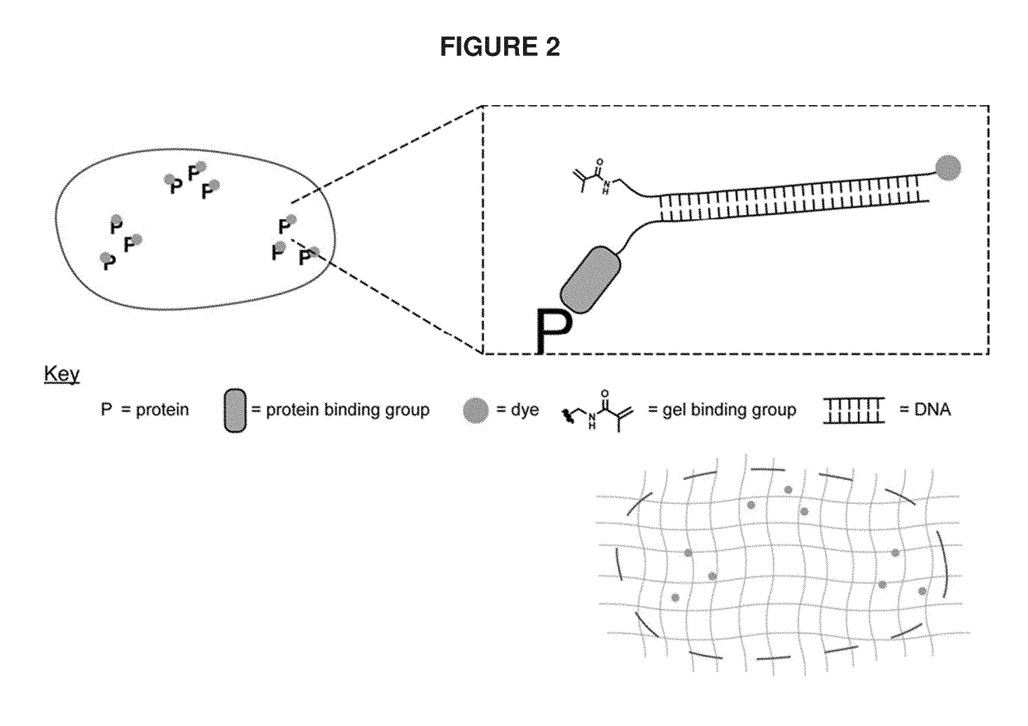

Critical to the success of the ExM process was the ability to physically connect the fluorescent protein-binding-groups to the polymer network. Current ExM attachment chemistry uses a trifunctional, double-stranded DNA linker to accomplish this. Because the tissue digestion enzymes are also capable of digesting the antibodies typically used as protein-binding-groups, it has been understood that the fluorescent dyes must be attached to the DNA and not the antibody. Also needed is the presence of a chemical group that can polymerize into the gel matrix (shown here as a methacrylamide group) on the DNA. Current examples of ExM use the chemical arrangement shown in FIG. 2 where one strand of DNA is connected to the protein-binding-group while the complementary strand possesses both the dye and the polymerizable group. Using this strategy, cells and brain tissue were successfully stained with up to 3 different protein-binding-groups, expanded, and imaged. However, because the number of fluorescent dyes that can be used is small (typically <6), this strategy is limited to imaging only a small number of proteins per sample. Additionally, the polymerization process dampens the fluorescence of the dyes which are permanently connected to the gel matrix. Alternative bioconjugation strategies have also been utilized to overcome some of these drawbacks. By rearranging the location of the three chemical groups (dye, gel binding group, and protein-binding-group) on the DNA linker, some previous limitations in protein imaging have been overcome.

However, using DNA/antibody conjugates has several disadvantages. Buffers with uncommon additives are necessary in order to prevent the DNA on the antibody from binding to the nuclear DNA in the sample. Also, the presence of the DNA on the antibody reduces the extent and the rate at which it binds to the target. The result is that the current ExM processes are lengthy, and the staining is commonly dim compared to controls. Surprisingly, it has been found not only that antibodies can be directly acrylated (and hence suitable for polymerization), either before, after, or at the same time as attachment with a detectable label, but also that the detectable label will remain after the tissue digestion step, which is necessary for ExM. Because the ExM digestion enzyme can possibly degrade antibodies, it had been assumed previously that all label detection would disappear with digestion. However, the present invention, called turbo-expansion microscopy (TurboExM), does not use DNA as a linker, stains samples brightly, and is more rapid than previous ExM processes.

In one aspect, provided herein are methods of labeling a biological sample, the methods comprising: contacting the sample with at least one binding composition under conditions where it selectively recognizes a target biomolecule, wherein the binding composition comprises a modified antibody comprising a first antibody having an antigen-binding site having an affinity for the target biomolecule, wherein the first antibody is operably linked to (i) a detectable label, and (ii) a polyelectrolyte gel binding moiety; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; and dialyzing the sample to expand the polyelectrolyte gel. In some embodiments, the methods further comprise the step of removing the binding composition unbound to the polyelectrolyte gel after covalently conjugating the polyelectrolyte gel binding moiety to it. In some embodiments, the modified antibody comprises a secondary antibody. In some embodiments, the first antibody comprises a polyclonal antibody. In some embodiments, the first antibody comprises a monoclonal antibody. In some embodiments, the first antibody is a secondary antibody. In some embodiments, the label and/or the polyelectrolyte gel binding moiety is operably linked to a constant region of the first antibody. In some embodiments, either the label or the polyelectrolyte gel binding moiety is/are operably linked to a Cγ2 or a Cγ3 region of a heavy chain of the first antibody. In some embodiments, the label is operably linked to either the Cγ2 or the Cγ3 region of a first heavy chain of the first antibody and the polyelectrolyte gel binding moiety is operably linked to the Cγ2 or the Cγ3 region of a second heavy chain of the first antibody. In some embodiments, the label and/or the polyelectrolyte gel binding moiety is operably linked at the location of one or more disulfide linkages with the antibody. In some embodiments, the label and the polyelectrolyte gel binding moiety are operably linked to the first antibody either simultaneously or sequentially in either order.

In another aspect, provided herein are methods of labeling a biological sample, the methods comprising: contacting the sample with at least one binding composition under conditions where it selectively recognizes a target biomolecule, wherein the binding composition comprises a modified antigen-binding fragment comprising a first antigen-binding fragment having an antigen-binding site having an affinity for the target biomolecule, wherein the first antigen-binding fragment is operably linked to (i) a detectable label, and (ii) a polyelectrolyte gel binding moiety; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; and dialyzing the sample to expand the polyelectrolyte gel. In some embodiments, the methods further comprise the step of removing the binding composition unbound to the polyelectrolyte gel after covalently conjugating the polyelectrolyte gel binding moiety to it. In some embodiments, the modified antibody comprises a secondary antigen-binding fragment. In some embodiments, the first antigen-binding fragment is derived from a polyclonal antibody. In some embodiments, the first antigen-binding fragment is derived from a monoclonal antibody. In some embodiments, the first antigen-binding fragment is selected from the group consisting of a Fab, a Fab′, a (Fab′)₂, a F(ab′)2, a Fv, a single chain antibody (SCA), and a scFv-Fc. In some embodiments, the label and/or the polyelectrolyte gel binding moiety is/are operably linked to a constant region of the first antigen-binding fragment. In some embodiments, the label and/or the polyelectrolyte gel binding moiety is operably linked at the location of one or more disulfide linkages with the antibody. In some embodiments, the label and the polyelectrolyte gel binding moiety are operably linked to the first antigen-binding fragment either simultaneously or sequentially in either order.

With respect to any one of the various aspects of the methods provided herein, in some embodiments, the methods further comprise the step of removing the binding composition unbound to the polyelectrolyte gel after covalently conjugating the polyelectrolyte gel binding moiety to it. In some embodiments, the modified antibody comprises a secondary antibody. In some embodiments, the target biomolecule comprises a target antibody or a target antigen-binding fragment, or the target antibody may comprise a secondary antibody. In some embodiments, the affinity of the antigen-binding site for the target biomolecule is a high affinity with an affinity constant (Kₐ) greater than 10⁴ M⁻¹ or in the range of 10⁵-10¹¹ M⁻¹. In some embodiments, the binding composition is a specific binding composition having a dissociation constant (K_{D}) less than about 1×10⁻⁵ M or less than about 1×10⁻⁶ M or less than about 1×10⁻⁷ M. In some embodiments, the polyelectrolyte gel binding moiety is a methacryloyl group. In some embodiments, the monomer solution comprises sodium acrylate, acrylamide, and N-N′-methylenebisacrylamide. In some embodiments, the free radical polymerization is induced with ammonium persulfate (APS) initiator and tetramethylethylenediamine (TEMED). In some embodiments, the biological is chemically fixed and permeabilized prior to contact with the binding composition. In some embodiments, dialyzing the sample to expand the polyelectrolyte gel comprises dialyzing the sample in water to expand the polyelectrolyte gel. In some embodiments, the label is a detectable label. In some embodiments, the label is a fluorophore.

In another aspect, provided herein are methods of imaging a biological sample, the methods comprising labeling the sample via any one of the methods as described herein and obtaining an image of the sample after expanding the polyelectrolyte gel. In some embodiments, the methods of imaging a biological sample further comprise obtaining an image of the sample before expanding the polyelectrolyte gel.

In another aspect, provided herein are methods of analyzing a biological sample, the methods comprising the steps of: contacting the sample with a set of modified antibodies that selectively recognize a set of target biomolecules under conditions where the modified antibodies selectively recognize the target biomolecules, wherein each modified antibody comprises an antibody comprising an antigen-binding site having an affinity specific for one of the target biomolecules, wherein the antibody is operably linked to (i) a detectable label distinct to that antibody, and (ii) a polyelectrolyte gel binding moiety; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; dialyzing the sample to expand the polyelectrolyte gel; and for each target biomolecule, detecting the label distinct for the antibody having an affinity specific for that target biomolecule. In some embodiments, the label operably linked to the antibody for a plurality of the target biomolecules includes a fluorophore that is common for that plurality of targets, and each antibody of that plurality is hybridized separately from the other probes of that plurality and is removed following detection of that label. In some embodiments, the plurality of target biomolecules is the set of target biomolecules. In some embodiments, the target biomolecules comprise target antibodies or target antigen-binding fragments. In some embodiments, the target antibody is a secondary antibody. In some embodiments, the label is detected by confocal microscopy.

In another aspect, provided herein are methods of analyzing a biological sample, the methods comprising the steps of: contacting the sample with a set of modified antigen-binding fragments that selectively recognize a set of target biomolecules under conditions where the modified antigen-binding fragments selectively recognize the target biomolecules, wherein each modified antigen-binding fragment comprises an antigen-binding fragment comprising an antigen-binding site having an affinity specific for one of the target biomolecules, wherein the antigen-binding fragment is operably linked to (i) a detectable label distinct to that antigen-binding fragment, and (ii) a polyelectrolyte gel binding moiety; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; dialyzing the sample to expand the polyelectrolyte gel; and for each target biomolecule, detecting the label distinct for the antigen-binding fragment having an affinity specific for that target biomolecule. In some embodiments, the label operably linked to the antigen-binding fragment for a plurality of the target biomolecules includes a fluorophore that is common for that plurality of targets, and each antigen-binding fragment of that plurality is hybridized separately from the other probes of that plurality and is removed following detection of that label. In some embodiments, the plurality of target biomolecules is the set of target biomolecules. In some embodiments, the target biomolecules comprise target antibodies or target antigen-binding fragments. In some embodiments, the target antibody is a secondary antibody. In some embodiments, the label is detected by confocal microscopy.

In another aspect, provided herein are kits for modifying an antibody or an antigen-binding fragment, the kits comprising: a first reagent comprising a label; a second reagent comprising a polyelectrolyte gel binding moiety. In some embodiments, the kits further comprise a first antibody comprising an antigen-binding site having an affinity for a target biomolecule. In some embodiments, the kits further comprise a first antigen-binding fragment comprising an antigen-binding site having an affinity for a target biomolecule. In some embodiments, the label comprises a fluorophore.

In another aspect, provided herein are kits for labeling a biological sample, the kit comprising: a first reagent comprising a label; a second reagent comprising a polyelectrolyte gel binding moiety; and a protease. In some embodiments, the kits further comprise a first antibody comprising an antigen-binding site having an affinity for a target biomolecule. In some embodiments, the kits further comprise a first antigen-binding fragment comprising an antigen-binding site having an affinity for a target biomolecule. In some embodiments, the kits further comprise monomers of a polyelectrolyte gel. In some embodiments, the monomers comprise sodium acrylate, acrylamide or a combination thereof. In some embodiments, the kits further comprise a cross-linking reagent. In some embodiments, the cross-linking reagent comprises N-N′-methylenebisacrylamide. In some embodiments, the kits further comprise ammonium persulfate (APS) and/or tetramethylethylenediamine (TEMED). In some embodiments, the kits further comprise a detection reagent specific for the label. In some embodiments, the label comprises a fluorophore. In some embodiments, the protease comprises Proteinase K. In some embodiments, the kits further comprise a physiological buffer.

Typically, the acrylate is a polymerizable group, therefore when polymerization occurs around it, it becomes attached to the growing polymer networks. One possible example of a reagent used to attached the polymerizable groups is Acryoyl-X (ThermoFisher).

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In each pair, the light and heavy chain variable regions (V_{L} and V_{H}) are together responsible for binding to an antigen, and the constant regions (C_{L}, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains V_{H}, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to V_{H}, Cγ1, Cγ2, Cγ3, V_{L}, and C_{L}.

Depending on the amino acid sequence of the constant domain of their heavy chains, intact antibodies can be assigned to different “classes.” There are five-major classes (isotypes) of intact antibodies: IgA, IgD, IgE, IgG, and IgM, and several of these may be further divided into “subclasses,” e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art. While some antibodies are monomeric, most are multimers. As is well-known in the art, the subunits of most multimeric antibodies are linked to each other via disulfide bonds. For example, human IgG is comprised of two light chains and two heavy chains, with the two heavy chains typically linked by two disulfide bonds in the hinge region and with each light chain linked to a different heavy chain via a disulfide bond.

An antibody (Ab) is a protein that binds specifically to a particular substance, known as an “antigen” (Ag) (see below). An “antibody” or “antigen-binding fragment” is an immunoglobulin that binds a specific “epitope.” The term encompasses pollyclonal, monoclonal, and chimeric antibodies (e.g., multispecific antibodies). In nature, antibodies are generally produced by lymphocytes in response to immune challenge, such as by infection or immunization. An “antibody combining site” is that structural portion of an antibody molecule comprised of heavy and light chain variable and hypervariable regions that specifically binds antigen.

The terms “antibody” or “antigen-binding fragment” respectively refer to intact molecules as well as functional fragments thereof, such as Fab, a scFv-Fc bivalent molecule, F(ab′)₂, and Fv that are capable of specifically interacting with a desired target. In some embodiments, the antigen-binding fragments comprise:
(1) Fab, the fragment which contains a monovalent antigen-binding fragment of an antibody molecule, which can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)2 is a dimer of two Fab′ fragments held together by two disulfide bonds;
(4) Fv, a genetically engineered fragment containing the variable region of the light chain and the variable region of the heavy chain expressed as two chains;
(5) Single chain antibody (“SCA”), a genetically engineered molecule containing the variable region of the light chain and the variable region of the heavy chain, linked by a suitable polypeptide linker as a genetically fused single chain molecule; and
(6) scFv-Fc, is produced by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tandem scFv, a scFv-Fc bivalent molecule, an Fab, Fab′, Fv, F(ab′)₂ or an antigen binding scaffold (e.g., affibody, monobody, anticalin, DARPin, Knottin, etc.).

An “antigen” (Ag) is any substance that reacts specifically with antibodies or T lymphocytes (T cells). An “antigen-binding site” is the part of an immunoglobulin molecule that specifically binds an antigen. Additionally, an antigen-binding site includes any such site on any antigen-binding molecule, including, but not limited to an MHC molecule or T cell receptor, but it can also include any substance against which an antibody or antigen-binding fragment has been raised, including artificially manufactured antigens and/or artifically manufactured antibodies or antigen-binding fragments.

The term “antigenic material” covers any substance that will eleicit an innate or adaptive immune reponse. As used herein, “a portion of antigenic material” covers any antigenic material or fragment thereof, which is capable of eliciting an innate or adaptive immune response, even if the fragment is an incomplete representation or subset of the antigenic material as a whole. It can include the minimal antigen sequence required to elicit a specific immune response.

An “epitope” or “antigenic determinant” is a structure, usually made up of, but not limited to, a short peptide sequence or oligosaccharide, that is specifically recognized or specifically bound by a component of the immune system. It is the site on an antigen recognized by an antibody.

An antibody or antigen-binding fragment to a specific “target biomolecule” specifically interacts with at least some component of that “target biomolecule.”

An “immunogen” is a substance capable of eliciting an immune response. Each immunoglobulin molecue can potentially bind a variety of antibodies directed at its unique features, or “idiotype,” which is comprised of a series of “idiotopes.” An “idiotope” is a single antigenic determinant on a variable region of an antibody or T cell receptor. It is the set of idiotopes on an antibody which comprise the idiotype that makes that antibody unique. The “dominant idiotype” is the idiotype found on the major fraction of antibodies generated in response to an antigen.

As used herein, the terms “binds” or “binding” or grammatical equivalents, refer to compositions, directly or indirectly, having affinity for each other. “Specific binding” is where the binding is selective between two molecules. A particular example of specific binding is that which occurs between an antibody and an antigen. Typically, specific binding can be distinguished from non-specific when the dissociation constant (K_{D}) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding. “Affinity” is defined as the strength of the binding interaction of two molecules, such as an antigen and its antibody, which is defined for antibodies and other molecules with more than one binding site as the strength of binding of the ligand at one specified binding site. Although the noncovalent attachment of a ligand to antibody is typically not as strong as a covalent attachment, “high affinity” is for a ligand that binds to an antibody or other molecule having an affinity constant (Kₐ) of greater than 10⁴ M⁻⁴ typically 10⁻⁵-10₁₁ M⁻¹; as determined by inhibition ELISA or an equivalent affinity determined by comparable techniques, such as Scatchard plots or using K_{d}/dissociation constant, which is the reciprocal of the Kₐ, etc.

In one embodiment, the antibody, antigen-binding fragment, or affinity tag binds its target with a K_{D} of 0.1 nM -10 mM. In one embodiment, the antibody, antigen-binding fragment, or affinity tag binds its target with a K_{D} of 0.1 nM -1 mM. In one embodiment, the antibody, antigen-binding fragment, or affinity tag binds its target with a K_{D} within the 0.1 nM range. In one embodiment, the antibody, antigen-binding fragment, or affinity tag binds its target with a K_{D} of 0.1-2 nM. In another embodiment, the antibody, antigen-binding fragment, or affinity tag binds its target with a K_{D} of 0.1-1 nM. In another embodiment, the antibody, antigen-binding fragment, or affinity tag binds its target with a K_{D} of 0.05-1 nM. In another embodiment, the antibody, antigen-binding fragment, or affinity tag binds its target with a K_{D} of 0.1-0.5 nM. In another embodiment, the antibody, antigen-binding fragment, or affinity tag its target with a K_{D} of 0.1-0.2 nM. In some embodiments, the antibody, antigen-binding fragment, or affinity tag bind its target directly. In some embodiments, the antibody, antigen-binding fragment, or affinity tag bind its target indirectly, for example, the antibody, antigen-binding fragment, or affinity tag is a secondary antibody that binds to an antibody bound to the target. “Specificity” refers to the ability of an antibody to discriminate between antigenic determinants. It also refers to the precise determinants recognized by a particular receptor or antibody. “Specificity” may be affected by the conditions under which the discrimination or recognition takes place (e.g., pH, temperature, salt concentration, and other factors known in the art).

A “peptide” is a compound of two or more subunit amino acids, amino acid analogs, or peptidomimetics. The subunits may be linked by peptide bonds or by other bonds (e.g., as esters, ethers, and the like). While the term “protein” encompasses the term “polypeptide,” a “polypeptide” may be less than a full-length protein. However, the terms “polypeptide” and “protein” are used herein interchangeably and refer to any polymer of amino acids (dipeptide or greater) linked through peptide bods or modified peptide bonds. Thus, the terms “polypeptide” and “protein” include oligopeptides, protein fragments, fusion proteins, and the like. It should be appreciate that the terms “polypeptide” and “protein” can include moieties such as lipoproteins and glycoproteins, except where the context dictates otherwise.

A “domain” is a region of a protein or polypeptide having a significant tertiary structure.

“Conservatively modified variants” of domain sequences may also be envisioned. With respect to particular nucleic acid sequences, conservatively modified varients refers to those nucleic acids which encode identical or essentially identical amino acid sequences, or where the nucleic acid does not encode an amino acid sequence, to essentially identical sequences. Specifically, degenerate codon substitutions can be achieved by generating sequences in which the third position of one or more selected (or all) codons is substituted with mixed base and/or deoxyinosine or other modified residues. Alternatively, one or more amino acids may be substituted with an amino acid having a similar structure, activity, charge, or other proterty. Conservative substitution tables providing functionally similar amino acids are well-known in the art (see, e.g., Proc. Natl. Acad. Sci. USA 89: 10915-10919(1992)).

As used herein, “expression” refers to the process by which polynucleotides are transcribed into mRNA and/or translated into peptides, polypeptides, or proteins. If the polynucleotide is derived from genomic DNA, expression may include, but is not required to include, splicing of the mRNA transcribed from the genomic DNA, capping of the 5′ end of the mRNA, polyadenylation of the 3′ end of the mRNA, or other processing modifications or events.

A “tag peptide sequence” is a short peptide or poplypeptide chain of 3 or more amino acids, which is attached to an antibody or other protein or moiety of interest. In some embodiments, a polypeptide, protein, or chimeric protein comprises a tag polypeptide sequence, which is used for purification, detection, labeling or some other function, such as by specific binding to an antibody. The antibody may be in solution or bound to a surface. The tag peptide sequence should not interfere with the function of the rest of the polypeptide, protein, or chimeric protein. Examples of tag proteins are well-known to those of ordinary skill in the art.

The word “label” as used herein refers to a compound or composition which is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition, which is detectable.

As used herein, the term “probe” refers to synthetic or biologically produced nucleic acids that are engineered to contain specific nucleotide sequences which hybridize under stringent conditions to target nucleic acid sequences.

As used herein, a “labeled probe,” “antibody operably linked to a label,” “antibody operably linked to a detectable label,” “antigen-binding fragment operably linked to a label,” antigen-binding fragment operably linked to a detectable label,” “nucleic acid probe operably linked to a detectable label,” or “nucleic acid strand operably linked to a detectable label” refer to a probe which is prepared with a marker moiety, “label” or “detectable label” for detection. The marker moiety should be linked in a place and manner so as not to interfere with, significantly/substantially decrease or inhibit, the binding or affinity of the probe to the target. For example, with respect to an antibody (or antigen-binding protein) operably linked to a label, the label should be attached to the antibody (or antigen-binding fragment) in such a manner as to prevent the label from inhibiting binding of the antibody (or antigen-binding fragment) to its target biomolecule. With respect to an antibody, the marker moiety is preferably attached to a constant region of the antibody, prefably to a Cγ2 or a Cγ3 region of a heavy chain. With respect to an antigen-binding fragment, the marker moiety is preferably attached to a constant region of the antigen-binding fragment. Alternatively, the label and/or the polyelectrolyte gel binding moiety is preferably operably linked at the location of one or more disulfide linkages with the antibody. With respect to a nucleic acid, the marker moiety is attached at either the 5′ end, the 3′ end, internally, or in any possible combination thereof. The preferred marker moiety is an identifying label, preferably a detectable label. Preferably, the detectable label is a fluorophore. In some embodiments, one probe may be attached to multiple marker moieties. In some embodiments, multiple types of probes are used, each type having a different marker moiety. The labeled probe may also be comprised of a plurality of different antibodies (or antigen-binding fragments) each labeled with one or more marker moieties. Each of the marker moieties may be the same or different. It may be beneficial to label the different probes (e.g., antibodies or antigen-binding fragments) each with a different marker moiety. This can be accomplished by having a single distinguishable moiety on each probe. For example, probe A may be attached to moiety X and probe B may be attached to moiety Y. Alternatively, probe A may be attached to moieties X and Y while probe B may be attached to moiety Z and W. As another alternative, probe A may be attached to moieties X and Y while probe B may be attached to moieties Y and Z. All the probes “A” and “B” described above would be distinguishable and uniquely labeled.

“Acrylates” or “polyacrylates” are a family of polymers made from acrylate monomers, which are esters having vinyl groups. Acrylate monomers include, but are not limited to acrylamide, N-sioproylacrulamide, dimethylacrylamide, acrylic acid, methacrylic acid, hydroxyl ethyl acrylamide, or oligo(ethylene glycol) methyl ether methacrylate, which can polymerize. For example, free radical polymerization of an acrylate monomer solution comprising sodium acrylate, acrylamide and N-N′-methylenebisacrylamide can be induced by the addition of ammonium persulfate (APS) initiator and tetramethylethylenediamine (TEMED).

In the present invention, the antibody or antigen-binding fragment can be acrylated directly, making it suitable for polymerization. This process can be performed either before, after, or simultaneously with attachment of the detectable label (e.g., a fluorophore). The most staightforward way to acrylate antibodies is to use a reagent which can react with the many amino groups present on its surface such as the commercially available SE, 6-((acryloyl)amino)hexanoic acid, succinimidyl ester (Acryoyl-X, ThermoFisher A20770). Once the polymerizable group is presented on the surface of the antibody, free radical poymerization in its presence will result with it being attached to the polymer gel.

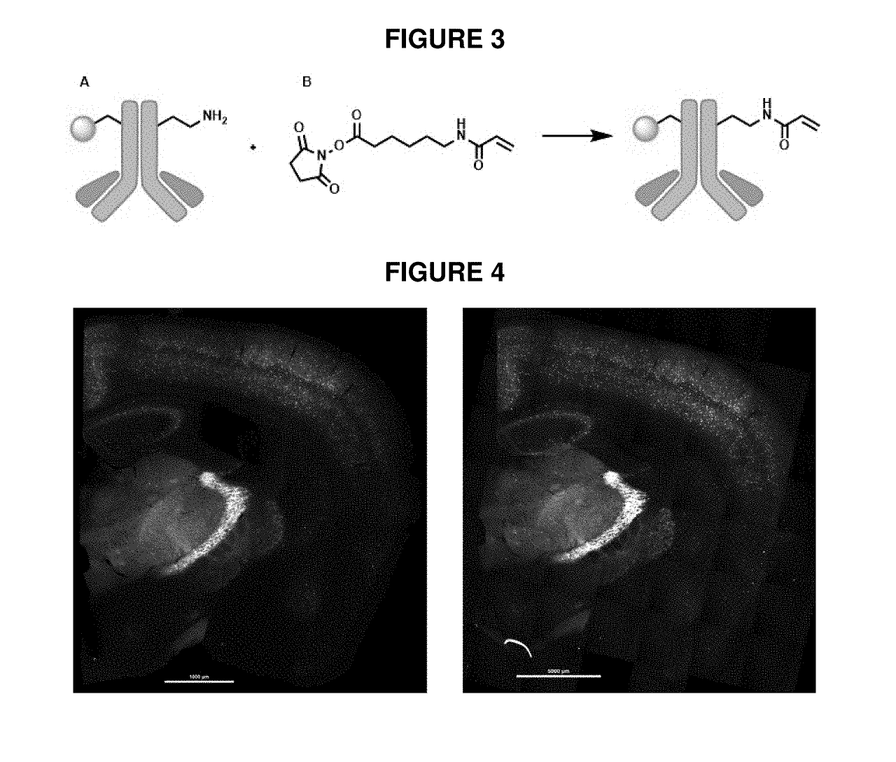

Direct acrylation of the antibody or antigen-binding fragment yields a “polyelectrolyte gel binding moiety” operably linked to the antibody or antigen-binding fragment. In some embodiments, the polyelectrolyte gel binding moiety is a acrylamide, methacrylamide, acrylate, or methacrylate group. For example, as shown in FIG. 3, the reagent is SE, 6-((acryloyl)amino)hexanoic acid, succinimidyl ester (Acryoyl-X, ThermoFisher A20770).

During free radical polymerization of the acrylate monomers (above), the “polyelectrolyte gel binding moiety” is covalently conjugated to the polyelectrolyte gel, thereby indirectly attaching the labeled antibody or antigen-binding fragment to the resulting polyelectrolyte gel. The “polyelectrolyte gel binding moiety” should be linked to the antibody or antigen-binding fragment in a place and manner so as not to interfere with, significantly/substantially decrease or inhibit, the binding or affinity of the probe to the target and also so as not to interfere with, significantly/substantially decrease or inhibit, the detection of the marker moiety. For example, with respect to an antibody (or antigen-binding protein) operably linked to a polyelectrolyte gel binding moiety, the polyelectrolyte gel binding moiety should be attached to the antibody (or antigen-binding fragment) in such a manner as to prevent the polyelectrolyte gel binding moiety from inhibiting binding of the antibody (or antigen-binding fragment) to its target biomolecule and should also be attached to the antibody (or antigen-binding fragment) in such a manner as to prevent the polyelectrolyte gel binding moiety from inhibiting detection of the label. With respect to an antibody, polyelectrolyte gel binding moiety is preferably attached to a constant region of the antibody, prefably to a Cγ2 or a Cγ3 region of a heavy chain. With respect to an antigen-binding fragment, the polyelectrolyte gel binding moiety is preferably attached to a constant region of the antigen-binding fragment. As shown in the Example in FIG. 3, in some embodiments, the modified antibody comprises an antibody modified with a fluorophore operably linked to a constant region on one heavy chain and a polyelectrolyte gel binding moiety operably linked to a constant region on the other heavy chain.

In some embodiments, pyridazinediones (PD), such as a dibromopyridazinedione (diBrPD), which contain both the acrylate group and the dye, can be inserted into one or more of the disulfide linkage(s) within the antibody. (See, e.g., Maruani et al., Nature Commun. 6:6645 [2015] [DOI: 10/1038/ncomms7645].) With this approach, the number and location of modification sites are controlled, the solubility of the antibody undergoes little or no alteration, and the reagents maintain the structural stability of the disulfide bond.

“Biological sample” includes sample of organs, tissues, cells, blood, fluid, or other materials obtained from a biological organism. It also includes a biological organism, a cell, virus, or other replicative entity. Also included are solid cultures (including bacterial or tissue cultures). Also included are solid sample, including, but not limited non-biological solids containing a biological organism, cell, virus, or other replicative entity; organs; tissues; cells; or sections (e.g., sagittal sections, cross-sections, and the like), washings, homogenizations, sonications, and similar treatments of biological samples. A biological sample may be obtained directly from a biological organism (e.g., a human or non-human animal, a plant, a fungus, a yeast, a protist, a bacterium or algae), it may be from a culture, or it may initially be attached to a non-biological solid. A biological sample may include a cancerous or noncancerous tumor or other growth, including a noncancerous aberrant growth.

A “physiological condition” of a biological organism may be normal or abnormal. The physiological condition may result from the genetic make-up of the organism (including, but not limited to, the expression of various proteins), from environmental factors (including, but not limited to, the ingestion of drugs, poisons, food, and beverages and the exposure of an organism to toxic or non-toxic substances), from disease (both infectious or non-infectious), from an injury, from a metabolic disorder, from pregnancy or nursing, and from a wide range of other circumstances, including genetic diseases, syndromes, and polymorphisms with respect to the genotype and/or phenotype of the organism, organ, tumor, tissue, or cell.

By “tissue sample” is meant a collection of similar cells obtained from a tissue of a subject or patient, preferably containing nucleated cells with chromosomal material. The four main human tissues are (1) epithelium; (2) the connective tissues, including blood vessels, bone and cartilage; (3) muscle tissue; and (4) nerve tissue. The source of the tissue sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. The tissue sample may contain compounds which are not naturally intermixed with the tissue in nature such as preservatives, anticoagulants, buffers, fixatives, nutrients, antibiotics, or the like.

For the purposes herein a “section” of a tissue sample is meant a single part or piece of a tissue sample, e.g., a thin slice of tissue or cells cut from a tissue sample. It is understood that multiple sections of tissue samples may be taken and subjected to analysis. Types of sections include sagittal sections and cross-sections and may be individual or serial.

As used herein, “cell line” refers to a permanently established cell culture that will proliferate given appropriate fresh medium and space.

### Detection Methods

In various aspects, provided herein are methods of detecting or locating a target in a biological sample. Targets are detected by contacting a biological sample with a target detection reagent, e.g., an antibody or fragment thereof, and a labeling reagent. The presence or absence of targets are detected by the presence or absence of the labeling reagent, and the location of the labeling reagent indicates where the target biomolecules were located. In some instances, the biological sample is contacted with the target detection reagent and the labeling reagent concurrently e.g., the detection reagent is a primary antibody and the labeling reagent is an operably linked fluorescent dye. Alternatively, the biological sample is contacted with the target detection reagent and the labeling reagent sequentially, e.g., the detection reagent is a primary antibody and the labeling reagent includes a secondary antibody. For example, the biological sample is incubated with the detection reagent, in some cases together with the labeling reagent, under conditions that allow a complex between the detection reagent (and labeling reagent) and target to form. After complex formation the biological sample is optionally washed one or more times to remove unbound detection reagent (and labeling reagent). When the biological sample is further contacted with a labeling reagent that specifically binds the detection reagent that is bound to the target, the biological sample can optionally be washed one or more times to remove unbound labeling reagent. The presence or absence of the target, and if present its location, in the biological sample is then determined by detecting the labeling reagent.

The methods described herein provide for the detection of multiple targets in a sample.

Multiple targets are identified by contacting the biological sample with additional detection reagents followed by additional labeling reagent specific for the additional detection reagents using the methods described above. For example, each target is associated with an affinity tag operably linked to a nucleic acid with a sequence specific or barcode for that target. In some cases, sets or subsets of labeled probes are prepared with distinct labels, e.g., fluorophores that are distinguished by their emission spectra, e.g., one that emits in the green spectra and one that emits in the red spectra. The labeled probes can then be added simultaneously to the biological sample to detect multiple targets at once. Alternatively, sets or subsets of labeled probes are prepared with the same label. Each of the labeled probes can then be added sequentially to detect a specific target, with each labeled probe removed from the biological sample prior to the addition of the next labeled probe to detect multiple targets sequentially.

The detection moiety, i.e., detectable label, is a substance used to facilitate identification and/or quantitation of a target. Detection moieties are directly observed or measured or indirectly observed or measured. Detection moieties include, but are not limited to, radiolabels that can be measured with radiation-counting devices; pigments, dyes or other chromogens that can be visually observed or measured with a spectrophotometer; spin labels that can be measured with a spin label analyzer; and fluorescent moieties, where the output signal is generated by the excitation of a suitable molecular adduct and that can be visualized by excitation with light that is absorbed by the dye or can be measured with standard fluorometers or imaging systems, for example. The detection moiety can be a luminescent substance such as a phosphor or fluorogen; a bioluminescent substance; a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The detection moiety may also take the form of a chemical or biochemical, or an inert particle, including but not limited to colloidal gold, microspheres, quantum dots, or inorganic crystals such as nanocrystals or phosphors. The term detection moiety or detectable label can also refer to a “tag” or hapten that can bind selectively to a labeled molecule such that the labeled molecule, when added subsequently, is used to generate a detectable signal. For instance, one can use biotin, iminobiotin or desthiobiotin as a tag and then use an avidin or streptavidin conjugate of horseradish peroxidase (HRP) to bind to the tag, and then use a chromogenic substrate (e.g., tetramethylbenzidine) or a fluorogenic substrate such as Amplex Red or Amplex Gold (Molecular Probes, Inc.) to detect the presence of HRP Similarly, the tag can be a hapten or antigen (e.g., digoxigenin), and an enzymatically, fluorescently, or radioactively labeled antibody can be used to bind to the tag. Numerous labels are known by those of skill in the art and include, but are not limited to, particles, fluorescent dyes, haptens, enzymes and their chromogenic, fluorogenic, and chemiluminescent substrates, and other.

A fluorophore is a chemical moiety that exhibits an absorption maximum beyond 280 nm, and when covalently attached in a labeling reagent retains its spectral properties. Fluorophores include, without limitation; a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine, a carbocyanine, a carbostyryl, a porphyrin, a salicylate, an anthranilate, an azulene, a perylene, a pyridine, a quinoline, a borapolyazaindacene, a xanthene, an oxazine or a benzoxazine, a carbazine, a phenalenone, a coumarin, a benzofuran and benzphenalenone and derivatives thereof. As used herein, oxazines include resorufins, aminooxazinones, diaminooxazines, and their benzo-substituted analogs.

When the fluorophore is a xanthene, the fluorophore may be a fluorescein, a rhodol, or a rhodamine. As used herein, fluorescein includes benzo- or dibenzofluoresceins, seminaphthofluoresceins, or naphthofluoresceins. Similarly, as used herein rhodol includes seminaphthorhodafluors. Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position. Fluorophores include xanthene (rhodol, rhodamine, fluorescein and derivatives thereof) coumarin, cyanine, pyrene, oxazine and borapolyazaindacene. In addition, the fluorophore can be sulfonated xanthenes, fluorinated xanthenes, sulfonated coumarins, fluorinated coumarins and sulfonated cyanines. The choice of the fluorophore in the labeling reagent will determine the absorption and fluorescence emission properties of the labeling reagent. Physical properties of a fluorophore label include spectral characteristics (absorption, emission and stokes shift), fluorescence intensity, lifetime, polarization and photo-bleaching rate all of which can be used to distinguish one fluorophore from another.

Typically the fluorophore contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

Preferably the detection moiety is a fluorescent dye. Fluorescent dyes include, for example, Fluorescein, Rhodamine, Texas Red, Cγ2, Cγ3, Cγ5, Cγ0, Cγ0.5, Cγ1, Cγ1.5, Cγ3.5, Cγ7, VECTOR Red, ELF™ (Enzyme-Labeled Fluorescence), FluorX, Calcein, Calcein-AM, CRYPTOFLUORT™'S, Orange (42 kDa), Tangerine (35 kDa), Gold (31 kDa), Red (42 kDa), Crimson (40 kDa), BHMP, BHDMAP, Br-Oregon, Lucifer Yellow, Alexa dye family, N-(6-(7-nitrobenz-2-oxa-1,3-diazol-4-yl)amino)caproyl) (NBD), BODIPY™, boron dipyrromethene difluoride, Oregon Green, MITOTRACKER™ Red, DiOC7(3), DiIC18, Phycoerythrin, Phycobiliproteins BPE (240 kDa) RPE (240 kDa) CPC (264 kDa) APC (104 kDa), Spectrum Blue, Spectrum Aqua, Spectrum Green, Spectrum Gold, Spectrum Orange, Spectrum Red, NADH, NADPH, FAD, Infra-Red (IR) Dyes, Cyclic GDP-Ribose (cGDPR), Calcofluor White, Tyrosine and Tryptophan.

Many of fluorophores can also function as chromophores and thus the described fluorophores are also preferred chromophores.

In addition to fluorophores, enzymes also find use as detectable moieties. Enzymes are desirable detectable moieties because amplification of the detectable signal can be obtained resulting in increased assay sensitivity. The enzyme itself does not produce a detectable response but functions to break down a substrate when it is contacted by an appropriate substrate such that the converted substrate produces a fluorescent, colorimetric or luminescent signal. Enzymes amplify the detectable signal because one enzyme on a labeling reagent can result in multiple substrates being converted to a detectable signal. This is advantageous where there is a low quantity of target present in the sample or a fluorophore does not exist that will give comparable or stronger signal than the enzyme. However, fluorophores are most preferred because they do not require additional assay steps and thus reduce the overall time required to complete an assay. The enzyme substrate is selected to yield the preferred measurable product, e.g. colorimetric, fluorescent or chemiluminescence. Such substrates are extensively used in the art.

A preferred colorimetric or fluorogenic substrate and enzyme combination uses oxidoreductases such as horseradish peroxidase and a substrate such as 3,3′-diaminobenzidine (DAB) and 3-amino-9-ethylcarbazol-e (AEC), which yield a distinguishing color (brown and red, respectively). Other colorimetric oxidoreductase substrates that yield detectable products include, but are not limited to: 2,2-azino-bis(3-ethylbenzothiaz-oline-6-sulfonic acid) (ABTS), o-phenylenediamine (OPD), 3,3′,5,5′-tetramethylbenzidine (TMB), o-dianisidine, 5-amino salicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including Amplexe Red reagent and its variants and reduced dihydroxanthenes, including dihydrofluoresceins and dihydrorhodamines including dihydrorhodamine 123. Peroxidase substrates that are tyramides represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

Additional colorimetric (and in some cases fluorogenic) substrate and enzyme combination use a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy4-methylcoumarinyl phosphate (DiFMUP) fluorescein diphosphate, 3-0-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates.

Glycosidases, in particular β-galactosidase, β-glucuronidase and β-glucosidase, are additional suitable enzymes. Appropriate colorimetric substrates include, but are not limited to, 5-bromo4-chloro-3-indolyl β-D-galactopyranoside (X-gal) and similar indolyl galactosides, glucosides, and glucuronides, o-nitrophenyl β-D-galactopyranoside (ONPG) and p-nitrophenyl β-D-galactopyranosid-e. Preferred fluorogenic substrates include resorufin β-D-galactopyranoside, fluorescein digalactoside (FDG), fluorescein diglucuronide and their structural variants, 4-methylumbelliferyl β-galactopyrano side, carboxyumbelliferyl β-D-galactopyrano side and fluorinated coumarin β-D-galactopyrano sides.

Additional enzymes include, but are not limited to, hydrolases such as cholinesterases and peptidases, oxidases such as glucose oxidase and cytochrome oxidases, and reductases for which suitable substrates are known.

Enzymes and their appropriate substrates that produce chemiluminescence are preferred for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful. For example, the enzyme is luciferase or aequorin. The substrates are luciferine, ATP, Ca⁺⁺ and coelenterazine.

In addition to enzymes, haptens such as biotin are useful detectable moieties. Biotin is useful because it can function in an enzyme system to further amplify a detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, or nucleotides.

In some cases, a detectable moiety is a fluorescent protein. Exemplary fluorescent proteins include green fluorescent protein (GFP), the phycobiliproteins and the derivatives thereof, luciferase or aequorin. The fluorescent proteins, especially phycobiliprotein, are particularly useful for creating tandem dye labeled labeling reagents. These tandem dyes comprise a fluorescent protein and a fluorophore for the purposes of obtaining a larger stokes shift where the emission spectra is farther shifted from the wavelength of the fluorescent protein's absorption spectra. This is particularly advantageous for detecting a low quantity of a target in a sample where the emitted fluorescent light is maximally optimized, in other words little to none of the emitted light is reabsorbed by the fluorescent protein. For this to work, the fluorescent protein and fluorophore function as an energy transfer pair where the fluorescent protein emits at the wavelength that the fluorophore absorbs at and the fluorphore then emits at a wavelength farther from the fluorescent proteins than could have been obtained with only the fluorescent protein. A particularly useful combination is phycobiliproteins and sulforhodamine fluorophores, or the sulfonated cyanine fluorophores; or the sulfonated xanthene derivatives. Alternatively, the fluorophore functions as the energy donor and the fluorescent protein is the energy acceptor.

### Methods of Visualizing the Detection Moiety Depend on the Label.

In some cases, the sample is illuminated with a wavelength of light selected to give a detectable optical response, and observed with a means for detecting the optical response.

Equipment that is useful for illuminating fluorescent compounds of the present invention includes, but is not limited to, hand-held ultraviolet lamps, mercury arc lamps, xenon lamps, lasers and laser diodes. These illumination sources are optically integrated into laser scanners, fluorescent microplate readers or standard or microfluorometers. The degree and/or location of signal, compared with a standard or expected response, indicates whether and to what degree the sample possesses a given characteristic or desired target.

The optical response is optionally detected by visual inspection, or by use of any of the following devices: CCD camera, video camera, photographic film, laser-scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, examination of the sample optionally includes sorting portions of the sample according to their fluorescence response.

When an indirectly detectable label is used then the step of illuminating typically includes the addition of a reagent that facilitates a detectable signal such as colorimetric enzyme substrate.

Radioisotopes are also considered indirectly detectable wherein an additional reagent is not required but instead the radioisotope must be exposed to X-ray film or some other mechanism for recording and measuring the radioisotope signal. This can also be true for some chemiluminescent signals that are best observed after expose to film.

The term “subject” refers to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

As used in the specification and claims, the singular forms “a,” “an,” and “the” include plural references unless the context dictates otherwise. For example, the term “a molecule” can also include a plurality of molecules.

When not otherwise stated, “substantially” means “being largely, but not wholly, that which is specified.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value such as an amount, a temporal duration, a concentration, and the like, may encompass variations of ±20% or ±10%, more preferably ±5%, even more preferably ±1%, and still more preferably ±0.1% from the specified value, as such variations are appropriate to perform the disclosed methods.

Various aspects and embodiments of the present invention will now be described in more detail by way of example. These examples are intended merely to be illustrative of the present invention and are not intended to limit the invention in any way. It will be appreciated that modification of detail may be made without departing from the scope of the invention.

### EXAMPLES

### Example 1

A target biomolecule of interest is identified, and an antibody having an antigen-binding site with an affinity for the target biomolecule is obtained. The antibody is modified to be operably linked to a detectable label and also operably linked to a polyelectrolyte gel binding moiety to yield a modified antibody. The modified antibody is a binding composition or a component of the binding composition.

A biological sample of interest is obtained. The sample is contacted with the binding composition under conditions where it selectively recognizes the target biomolecule. The biological sample is incubated with the detection reagent, in some cases together with the labeling reagent, under conditions that allow a complex between the detection reagent (and labeling reagent) and target to form. Upon treatment with a solution of monomers and subsequent free radical polymerization, a polyelectrolyte gel is formed to which the polyelectroylte gel binding moiety (operably linked to the antibody) is covalently attached. After complex formation the biological sample is optionally washed one or more times to remove unbound detection reagent (and possibly labeling reagent). The sample is digested by proteolysis and dialyzed to expand the polyelectrolyte gel. When the biological sample is further contacted with a labeling reagent that specifically binds the detection reagent that is bound to the target, the biological sample can optionally be washed one or more times to remove unbound labeling reagent. The presence or absence of the target, and if present its location, in the biological sample is then determined by detecting the labeling reagent. An image of the sample is obtained after the polyelectrolyte gel has been expanded and optionally before expansion of the polyelectrolyte gel.

### Example 2

A target biomolecule of interest is identified, and an antigen-binding fragment having an antigen-binding site with an affinity for the target biomolecule is obtained. The antigen-binding fragment is modified to be operably linked to a detectable label and also operably linked to a polyelectrolyte gel binding moiety to yield a modified antigen-binding fragment. The modified antigen-binding fragment is a binding composition or a component of the binding composition.

A biological sample of interest is obtained. The sample is contacted with the binding composition under conditions where it selectively recognizes the target biomolecule. The biological sample is incubated with the detection reagent, in some cases together with the labeling reagent, under conditions that allow a complex between the detection reagent (and labeling reagent) and target to form. Upon treatment with a solution of monomers and subsequent free radical polymerization, a polyelectrolyte gel is formed to which the polyelectroylte gel binding moiety (operably linked to the antigen-binding fragment) is covalently attached. After complex formation the biological sample is optionally washed one or more times to remove unbound detection reagent (and possibly labeling reagent). The sample is digested by proteolysis and dialyzed to expand the polyelectrolyte gel. When the biological sample is further contacted with a labeling reagent that specifically binds the detection reagent that is bound to the target, the biological sample can optionally be washed one or more times to remove unbound labeling reagent. The presence or absence of the target, and if present its location, in the biological sample is then determined by detecting the labeling reagent. An image of the sample is obtained after the polyelectrolyte gel has been expanded and optionally before expansion of the polyelectrolyte gel.

### Example 3

As described in Example 1(above), a target biomolecule of interest (a primary anti-PV antibody to murine parvalbumin protein) was identified, and an antibody having an antigen-binding site with an affinity for the target biomolecule was obtained (here, a secondary antibody to the primary anti-PV antibody). This secondary antibody was modified to be operably linked to a fluorophore (an atto647N dye) and also operably linked to a polyelectrolyte gel binding moiety to yield a modified antibody, as shown in FIG. 3.

A mouse brain sample was obtained. The sample was first contacted with the primary anti-PV antibody and then contacted with the binding composition comprising the modified secondary antibody under conditions where it selectively recognizes the primary anti-PV antibody target biomolecule. In each case, the sample was incubated under conditions that allow a complex between the detection reagent (and labeling reagent) and target to form. Upon treatment with a solution of monomers and subsequent free radical polymerization, a polyelectrolyte gel was formed to which the polyelectrolyte gel binding moiety (operably linked to the secondary antibody) was covalently attached. At this stage the sample was digested with Proteinase K, a non-selective enzyme necessary to digest tissue enough for the gel to expand. After digestion, the sample was washed repeated with water to allow expansion. Importantly, the fluroescent signal from the antibody remained even after proteoloysis indicating that the novel antibody formulatio was robust enough to withstand the necessary digestion step.

As shown in FIG. 4, an image of the sample was obtained both after the polyelectrolyte gel has been expanded and before expansion of the polyelectrolyte gel. As shown by the scale bar in each panel, the right sample (scale bar =5000 μm) is four times larger than the left sample (scale bar =1000 μm). The expansion of the sample produced a sample which retained the staining pattern of the pre-expanded sample, nothwithstanding digestion of the sample via proteolysis.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method of labeling a biological sample, the method comprising:
contacting the sample with at least one binding composition under conditions where it selectively recognizes a target biomolecule, wherein the binding composition comprises a modified antibody comprising a first antibody having an antigen-binding site having an affinity for the target biomolecule, wherein the first antibody is operably linked to (i) a label, and (ii) a polyelectrolyte gel binding moiety;
contacting the sample with a solution comprising monomers of a polyelectrolyte gel;
by free radical polymerization, polymerizing the monomers to form the polyelectrolyte gel and covalently conjugate the polyelectrolyte gel binding moiety to the polyelectrolyte gel;
proteolytically digesting the sample; and
dialyzing the sample to expand the polyelectrolyte gel.

2. The method according to claim 1, further comprising the step of:
removing the binding composition unbound to the polyelectrolyte gel after covalently conjugating the polyelectrolyte gel binding moiety to it.

3. The method according to claim 1, wherein the modified antibody comprises a secondary antibody.

4. The method according to claim 1, wherein the target biomolecule comprises a target antibody or a target antigen-binding fragment.

5. The method according to claim 4, wherein the target antibody is a secondary antibody.

6. The method according to claim 1, wherein dialyzing the sample to expand the polyelectrolyte gel comprises dialyzing the sample in water to expand the polyelectrolyte gel.

7. The method according to claim 1, wherein the affinity of the antigen-binding site for the target biomolecule is a high affinity with an affinity constant (Kₐ) greater than 10⁴ M⁻¹.

8. The method according to claim 7, wherein the affinity constant (Kₐ) is 10⁵ −10¹¹ M⁻¹.

9. The method according to claim 1, wherein the binding composition is a specific binding composition having a dissociation constant (K_{D}) less than about 1×10⁻⁵ M.

10. The method according to claim 9, wherein the dissociation constant (K_{D}) is less than about 1×10⁻⁶ M.

11. The method according to claim 9, wherein the dissociation constant (K_{D}) is less than about 1×10⁻⁷ M.

12. The method according to claim 1, wherein the first antibody comprises a polyclonal antibody.

13. The method according to claim 1, wherein the first antibody comprises a monoclonal antibody.

14. The method according to claim 1, wherein the first antibody is a secondary antibody.

15. The method according to claim 1, wherein the label or the polyelectrolyte gel binding moiety is operably linked to a constant region of the antibody.

16. The method according to claim 1, wherein the label or the polyelectrolyte gel binding moiety is operably linked to a constant region of the first antibody.

17. The method of labeling a biological sample according to claim 1, wherein either the label or the polyelectrolyte gel binding moiety is operably linked to a Cγ2 or a Cγ3 region of a heavy chain of the first antibody.

18. The method of labeling a biological sample according to claim 17, wherein the label is operably linked to either the Cγ2 or the Cγ3 region of a first heavy chain of the first antibody and the polyelectrolyte gel binding moiety is operably linked to the Cγ2 or the Cγ3 region of a second heavy chain of the first antibody.

19. The method according to claim 1, wherein the first antibody comprises at least two chains with a disulfide linkage between the two chains and either the label or the polyelectrolyte gel binding moiety is operably linked to the first antibody at the disulfide linkage.

20. The method according to claim 1, wherein prior to contacting the sample with at least one binding composition, the label is operably linked to the antibody before the polyelectrolyte gel binding moiety is operably linked to the first antibody.

21. The method according to claim 1, wherein prior to contacting the sample with at least one binding composition, the label is operably linked to the first antibody after the polyelectrolyte gel binding moiety is operably linked to the first antibody.

22. The method according to claim 1, wherein prior to contacting the sample with at least one binding composition, the label is operably linked to the first antibody and simultaneously as the polyelectrolyte gel binding moiety is operably linked to the first antibody.

23. The method according to claim 1, wherein the polyelectrolyte gel binding moiety is a methacryloyl group.

24. The method according to claim 1, wherein the monomer solution comprises sodium acrylate, acrylamide, and N-N′-methylenebisacrylamide.

25. The method according to claim 1, wherein the polyelectrolyte gel binding moiety or the label comprises a dibromopyridazinedione.

26. The method according to claim 1, wherein the free radical polymerization is induced with ammonium persulfate (APS) initiator and tetramethylethylenediamine (TEMED).

27. The method according to claim 1, wherein the biological sample is chemically fixed and permeabilized prior to contact with the binding composition.

28. The method according to claim 1, wherein the label operably linked to the first antibody is a detectable label.

29. The method according to claim 1, wherein the label operably linked to the first antibody is a fluorophore.

30. A method of imaging a biological sample, the method comprising:
labeling the sample according to the method of any one of claims 1-29; and
obtaining an image of the sample after expanding the polyelectrolyte gel.

31. The method according to claim 30, wherein obtaining the image of the sample comprises detecting the label.

32. The method according to claim 30, wherein obtaining the image of the sample comprises detecting the label operably linked to the first antibody.

33. The method according to any one of claims 30-32, further comprising the step of: obtaining an image of the sample before expanding the polyelectrolyte gel.

34. The method according to claim 33, wherein obtaining the image of the sample before expansion comprises detecting the label.

35. The method according to any one of claims 30-34, wherein the image(s) is/are obtained by confocal microscopy.

36. A method of labeling a biological sample, the method comprising:
contacting the sample with at least one binding composition under conditions where it selectively recognizes a target biomolecule, wherein the binding composition comprises a modified antigen-binding fragment comprising a first antigen-binding fragment having an antigen-binding site having an affinity for the target biomolecule, wherein the first antigen-binding fragment is operably linked to (i) a label, and (ii) a polyelectrolyte gel binding moiety;
contacting the sample with a solution comprising monomers of a polyelectrolyte gel;
by free radical polymerization, polymerizing the monomers to form the polyelectrolyte gel and covalently conjugate the polyelectrolyte gel binding moiety to the polyelectrolyte gel;
proteolytically digesting the sample; and
dialyzing the sample in water to expand the polyelectrolyte gel.

37. The method according to claim 36, further comprising the step of:
removing the binding composition unbound to the polyelectrolyte gel after covalently conjugating the polyelectrolyte gel binding moiety to it.

38. The method according to claim 36, wherein the modified antigen-binding fragment comprises a secondary antigen-binding fragment.

39. The method according to claim 36, wherein the target biomolecule comprises a target antibody or a target antigen-binding fragment.

40. The method according to claim 39, wherein the target antibody is a secondary antibody.

41. The method according to claim 36, wherein the first antigen-binding fragment is derived from a polyclonal antibody.

42. The method according to claim 36, wherein the first antigen-binding fragment is derived from a monoclonal antibody.

43. The method according to claim 36, wherein the first antigen-binding fragment is selected from the group consisting of a Fab, a Fab′, a (Fab′)₂, a F(ab′)2, a Fv, a single chain antibody (SCA), and a scFv-Fc.

44. The method according to claim 36, wherein the label or the polyelectrolyte gel binding moiety is operably linked to a constant region of the first antigen-binding fragment.

45. The method according to claim 1, wherein the first antigen-binding fragment comprises at least two chains with a disulfide linkage between the two chains and either the label or the polyelectrolyte gel binding moiety is operably linked to the first antigen-binding fragment at the disulfide linkage.

46. The method according to claim 36, wherein dialyzing the sample to expand the polyelectrolyte gel comprises dialyzing the sample in water to expand the polyelectrolyte gel.

47. The method according to claim 36, wherein the affinity of the antigen-binding site for the target biomolecule is a high affinity with an affinity constant (Kₐ) greater than 10⁴ M⁻¹.

48. The method according to claim 47, wherein the affinity constant (Kₐ) is 10⁵−10¹¹ M ⁻¹.

49. The method according to claim 36, wherein the binding composition is a specific binding composition having a dissociation constant (K_{D}) less than about 1×10⁻⁵ M.

50. The method according to claim 49, wherein the dissociation constant (K_{D}) is less than about 1×10⁻⁶ M.

51. The method according to claim 49, wherein the dissociation constant (K_{D}) is less than about 1×10⁻⁷ M.

52. The method according to claim 36, wherein the polyelectrolyte gel binding moiety is a methacryloyl group.

53. The method according to claim 36, wherein the monomer solution comprises sodium acrylate, acrylamide, and N-N′-methylenebisacrylamide.

54. The method according to claim 36, wherein the polyelectrolyte gel binding moiety or the label comprises a dibromopyridazinedione.

55. The method according to claim 36, wherein the free radical polymerization is induced with ammonium persulfate (APS) initiator and tetramethylethylenediamine (TEMED).

56. The method according to claim 36, wherein the biological sample is chemically fixed and permeabilized prior to contact with the binding composition.

57. The method according to claim 36, wherein the label operably linked to the first antigen-binding fragment is a detectable label.

58. The method according to claim 36, wherein the label operably linked to the first antigen-binding fragment is a fluorophore.

59. A method of imaging a biological sample, the method comprising:
labeling the sample according to the method of any one of claims 36-58; and
obtaining an image of the sample after expanding the polyelectrolyte gel.

60. The method according to claim 59, wherein obtaining the image of the sample comprises detecting the label.

61. The method according to claim 59, wherein obtaining the image of the sample comprises detecting the label operably linked to the first antigen-binding fragment.

62. The method according to any one of claims 59-61, further comprising the step of:
obtaining an image of the sample before expanding the polyelectrolyte gel.

63. The method of claim 60, wherein obtaining the image of the sample before expansion comprises detecting the label.

64. The method according to any one of claims 59-63, wherein the image(s) is/are obtained by confocal microscopy.

65. A method of analyzing a biological sample, the method comprising:
(a) contacting the sample with a set of modified antibodies that selectively recognize a set of target biomolecules under conditions where the modified antibodies selectively recognize the target biomolecules, wherein each modified antibody comprises an antibody having an antigen-binding site having an affinity specific for one of the target biomolecules, wherein the antibody is operably linked to (i) a label distinct to that antibody, and (ii) a polyelectrolyte gel binding moiety;
(b) contacting the sample with a solution comprising monomers of a polyelectrolyte gel;
(c) by free radical polymerization, polymerizing the monomers to form the polyelectrolyte gel and covalently conjugate the polyelectrolyte gel binding moiety to the polyelectrolyte gel;
(d) proteolytically digesting the sample;
(e) dialyzing the sample to expand the polyelectrolyte gel; and
(f) for each target biomolecule, detecting the label distinct for the antibody having an affinity specific for that target biomolecule.

66. The method according to claim 65, wherein the label operably linked to the antibody for a plurality of the target biomolecules includes a fluorophore that is common for that plurality of targets, and each antibody of that plurality is hybridized separately from the other probes of that plurality and is removed following detection of that label.

67. The method according to claim 65, wherein the plurality of target biomolecules is the set of target biomolecules.

68. The method according to claim 65, wherein the target biomolecules comprise target antibodies or target antigen-binding fragments.

69. The method according to claim 68, wherein the target antibody is a secondary antibody.

70. The method according to claim 65, wherein dialyzing the sample to expand the polyelectrolyte gel comprises dialyzing the sample in water to expand the polyelectrolyte gel.

71. The method according to any one of claims 65-70, wherein the label is detected by confocal microscopy.

72. A method of analyzing a biological sample, the method comprising:
(a) contacting the sample with a set of modified antigen-binding fragments that selectively recognize a set of target biomolecules under conditions where the modified antigen-binding fragments selectively recognize the target biomolecules, wherein each modified antigen-binding fragment comprises an antigen-binding fragment having an antigen-binding site having an affinity specific for one of the target biomolecules, wherein the antigen-binding fragment is operably linked to (i) a label distinct to that antigen-binding fragment, and (ii) a polyelectrolyte gel binding moiety;
(b) contacting the sample with a solution comprising monomers of a polyelectrolyte gel;
(c) by free radical polymerization, polymerizing the monomers to form the polyelectrolyte gel and covalently conjugate the polyelectrolyte gel binding moiety to the polyelectrolyte gel;
(d) proteolytically digesting the sample;
(e) dialyzing the sample to expand the polyelectrolyte gel; and
(f) for each target biomolecule, detecting the label distinct for the antigen-binding fragment having an affinity specific for that target biomolecule.

73. The method according to claim 72, wherein the label operably linked to the antigen-binding fragment for a plurality of the target biomolecules includes a fluorophore that is common for that plurality of targets, and each antigen-binding fragment of that plurality is hybridized separately from the other probes of that plurality and is removed following detection of that label.

74. The method according to claim 72, wherein the plurality of target biomolecules is the set of target biomolecules.

75. The method according to claim 72, wherein the target biomolecules comprise target antibodies or target antigen-binding fragments.

76. The method according to claim 75, wherein the target antibody is a secondary antibody.

77. The method according to claim 72, wherein dialyzing the sample to expand the polyelectrolyte gel comprises dialyzing the sample in water to expand the polyelectrolyte gel.

78. The method according to any one of claims 72-77, wherein the label is detected by confocal microscopy.

79. A kit for modifying an antibody or an antigen-binding fragment, the kit comprising:
a. a first reagent comprising a label;
b. a second reagent comprising a polyelectrolyte gel binding moiety.

80. The kit according to claim 79, wherein the kit further comprises a first antibody having an antigen-binding site having an affinity for a target biomolecule.

81. The kit according to claim 79, wherein the kit further comprises a first antigen-binding fragment having an antigen-binding site having an affinity for a target biomolecule.

82. The kit according to claim 79, wherein the label comprises a fluorophore.

83. A kit for labeling a biological sample, the kit comprising:
a. a first reagent comprising a label;
b. a second reagent comprising a polyelectrolyte gel binding moiety; and
c. a protease.

84. The kit according to claim 83, wherein the kit further comprises a first antibody comprising an antigen-binding site having an affinity for a target biomolecule.

85. The kit according to claim 83, wherein the kit further comprises a first antigen-binding fragment comprising an antigen-binding site having an affinity for a target biomolecule.

86. The kit according to claim 83, wherein the kit further comprises monomers of a polyelectrolyte gel.

87. The kit according to claim 86, wherein the monomers comprise sodium acrylate, acrylamide or a combination thereof.

88. The kit according to claim 83, wherein the kit further comprises a cross-linking reagent.

89. The kit according to claim 88, wherein the cross-linking reagent comprises N-N′-methylenebisacrylamide.

90. The kit according to claim 88, wherein the first reagent or the second reagent comprises a dibromopyridazinedione.

91. The kit according to claim 83, wherein the kit further comprises ammonium persulfate (APS).

92. The kit according to claim 83, wherein the kit further comprises tetramethylethylenediamine (TEMED).

93. The kit according to claim 83, wherein the label comprises a fluorophore.

94. The kit according to claim 83, wherein the kit further comprises a detection reagent specific for the label.

95. The kit according to claim 83, wherein the protease comprises Proteinase K.

96. The kit according to claim 83, wherein the kit further comprises a physiological buffer.
